(19) United States
(12) Patent Application Publication
(10) Pub. No.: US 2008/0051613 A1
(43) Pub. Date: Feb. 28, 2008

(54) LUBRICANTS FOR USE IN PROCESSING OF METALLIC MATERIAL

(75) Inventors: Mami KATO, Kariya (JP); Teruo FUKAYA, Aichi-ken (JP)

Correspondence Address: GREENBLUM & BERNSTEIN, P.L.C., 1950 ROLAND CLARKE PLACE, RESTON, VA, 20191, US

(73) Assignee: TOYOTA BOSHOKU KABUSHIKI KAISHA, Aichi-ken (JP)

(21) Appl. No.: 11/845,461

(22) Filed: Aug. 27, 2007

(30) Foreign Application Priority Data
Aug. 28, 2006 (JP) 2006-230406

(51) Int. Cl. C07C 9/00

(52) U.S. Cl. 585/16

(57) ABSTRACT

A lubricant for use in press working of a metallic material includes a paraffinic hydrocarbon. The paraffinic hydrocarbon is contained at a rate of 96-100 wt % of total weight of the lubricant. The lubricant has a kinetic viscosity of 2.0 or less at 40° C. Preferably, the paraffinic hydrocarbon has a carbon number of 8-13 and a boiling point of 210° C. or less.

This application claims priority to Japanese patent application serial number 2006-230406, the contents of which are incorporated herein by reference.

### BACKGROUND OF THE INVENTION

The present invention relates to lubricants for use in processing (e.g., press working) of a metallic material (in particular, a rust-resistant steel plate), which processing is also referred to as metal processing. Further, the present invention relates to methods for processing the metallic material using the lubricants.

A rolled steel plate as a metallic material has been used in many fields for manufacturing cars, architectural materials, white goods, electronic devices or other such products. For example, the rolled steel plate for the cars must provide good rust-proof performance, which is important in a cold region in North America or North Europe, where rock salt (i.e., a corrosive material) is applied on the road as an anti-freezing agent in winter. Therefore, in recent years, there is an increased need for a rust-resistant rolled steel plate in the field for manufacturing cars. In Canada, an anti-corrosion code regulation was established in 1978. The Canadian anti-corrosion code regulation specified that a car body must have a resistance to surface rusting for five years and a resistance to perforation corrosion for ten years. Recently, the Canadian code regulation has generally been used as a standard for the rust-proof performance of the car body.

Examples of the rust-resistant steel plate may include an electrogalvanized steel plate, a hot dip galvanized steel plate, a zinc-nickel alloy electroplated steel plate and an organic composite plated steel plate. The electrogalvanized steel plate has been generally used in the field for manufacturing cars, because the electrogalvanized steel plate has good surface smoothness, easy weldability, easy coatability, good workability and inexpensive availability.

Generally, in the fields for manufacturing cars, architectural materials, white goods, electronic devices or other such articles, the metallic material, e.g., the rolled steel plate, is processed or formed to a desired shaped product by various processing. Examples of known metal processing techniques may include press working, e.g., press forming, blanking, fine blanking, piercing, bending, burring, drawing, trimming and crimping, each of which can be performed by means of a special processing tool.

In processing of the metallic material, lubricants are generally applied between the metallic material and the processing tool, e.g., the mold (the punch and die assembly), in order to reduce friction therebetween. The lubricants thus applied may effectively prevent the processing tool (the mold) from wearing by the friction, thereby extending a service life of the processing tool. In addition, the lubricants may effectively increase processing accuracy of the metallic material. Conventionally, the lubricants include additives, e.g., an extreme pressure agent, an oil-based agent and a rust inhibitive agent, in order to increase lubricity thereof. However, because the lubricants are generally nonvolatile, the lubricants may leave nonvolatile lubricant residues on the metallic material (a formed product) after the metallic material is processed. Therefore, the lubricant residues must be removed or washed out from the formed product before the processed metallic material is transferred to a next processing step. That is, in the metal processing using the conventional lubricants, it is essential to provide a washing step in order to remove the lubricant residues. However, it is preferable to omit the washing step from a point of view of environmental concerns and production efficiency. Therefore, there is a need to develop improved or volatile (quick-drying) lubricants that can provide substantially the same lubricity as the conventional nonvolatile lubricants.

Some quick-drying lubricants for use in metal processing have been developed. The known quick-drying lubricants may generally contain a volatile lubricant base that can be naturally evaporated within hours or days at ambient temperature and pressure. The quick-drying lubricants thus formulated may omit the washing step, because the nonvolatile lubricant residues are not substantially left on the metallic material after the metallic material is processed.

However, some of the known quick-drying lubricants do not have sufficient lubricity. The insufficient lubricity may cause cracking and galling in the formed product. In addition, the insufficient lubricity cannot sufficiently reduce the friction between the metallic material and the processing tool. As a result, the service life of the processing tool cannot be sufficiently extended. In order to increase the lubricity of the quick-drying lubricants, in some of the known quick-drying lubricants, the amount of the extreme pressure agent, the oil-based agent and the rust inhibitive agent is considerably increased. However, the increased amount of ingredients may produce undesirable residues on the metallic material after the metallic material is processed. That is, the lubricants thus formulated may have reduced self-removability. The residues remaining on the processed metallic material may cause negative effects in a post-processing step (e.g., a coating step).

Further, some of the known quick-drying lubricants contain chlorine ingredients. However, the chlorine ingredients contained in the lubricant can be easily decomposed to produce undesirable decomposition products during processing or with time. The decomposition products thus produced may rust the metallic material and the processing tool (the mold). Further, the chlorine ingredients may produce harmful or toxic substances when the lubricants are incinerated. Also, the chlorine ingredients may corrode or damage incinerators. In order to solve these problems, there is a need to develop improved or nonchlorine quick-drying (volatile) lubricants that can provide substantially the same lubricity as the nonvolatile lubricants.

Up to now some quick-drying nonchlorine lubricants for use in metal processing have been developed. For example, Japanese Laid-open Patent Publication Number 60-19952 teaches a quick-drying nonchlorine lubricative composition for use in metal processing, which composition includes a halogenated hydrocarbon having a boiling point of 23-125° C. and a fluorine containing oil having a boiling point of 130-250° C. Also, Japanese Laid-open Patent Publication Number 7-283353 teaches a quick-drying nonchlorine lubricative composition, which composition includes an isoparaffinic hydrocarbon having viscosity of 1.5-2.0, specific gravity of 0.75-0.76 and a kauri-butanol value of 27-28. In this composition, the isoparaffinic hydrocarbon may preferably be contained at a rate of 70% or more. This lubricative composition can evaporate within hours or days at ambient temperature. In addition, Japanese Laid-open Patent Publication Number 9-255975 also teaches a quick-drying nonchlorine lubricative composition, which composition includes a paraffinic hydrocarbon having a boiling point of 150-250° C. at ambient pressure and having a carbon number of 8-22 and an α olefin having a boiling point of 200-290° C. In this composition, the α olefin may preferably be contained at a rate of 10 wt % or more. However, none of the known quick-drying nonchlorine lubricants provides excellent lubricant functionality for use in metal processing in view of the environmental concerns and the self-removability.

Thus, there is a need in the art for an improved lubricant for use in processing of a metallic material.

### BRIEF SUMMARY OF THE INVENTION

In one embodiment of the present invention, a lubricant is taught for use in press working of a metallic material. The lubricant contains a paraffinic hydrocarbon at a rate of 96-100 wt % of total weight of the lubricant and has a kinetic viscosity of 2.0 or less at 40° C.

The present lubricant thus formulated can be effectively prevented from excessively adhering to the metallic material because of increased flowability thereof that is due to the low kinetic viscosity thereof. In addition, the lubricant can naturally and rapidly evaporate from the metallic material at ambient temperature and pressure because of increased volatility thereof that is due to a high containing rate of the volatile paraffinic hydrocarbon. As a result, the present lubricant does not substantially produce undesirable lubricant residues on the metallic material after the metallic material is press worked. Therefore, it is not necessary to provide a washing step in order to remove the lubricant residues from the press worked metallic material. In other words, the press worked metallic material can be further processed (e.g., plated or coated) directly without passing through the washing step. Further, according to the present lubricant, the metallic material having various thicknesses can be press worked without producing cracking and galling.

The paraffinic hydrocarbon having a carbon number of 8-13 and a boiling point of 210° C. or less may preferably be used. The lubricant using such a paraffinic hydrocarbon may have further increased volatility and flowability. Therefore, the lubricant can be further quickly evaporated or dried at ambient temperature and pressure. Generally, the lubricant containing such a paraffinic hydrocarbon may be evaporated within 24 hours, which time period corresponds to a maximum permitted time period in which the press worked metallic material must be transferred to a next processing step (e.g., a plating or coating step) in a production line. As a result, it is not necessary to stop the production line in order to dry the press worked metallic material.

Further, in another embodiment of the present invention, a method is taught for press working a metallic material using a processing tool. The method includes the steps of feeding the lubricant that is described above between the metallic material and the processing tool.

According to the present method, friction between the metallic material and the processing tool can be effectively reduced, so that the processing tool can effectively be prevented from wearing. As a result, the processing tool may have a long service life. Further, formation of cracking and galling on a press worked surface of the metallic material can be prevented.

Other objects, features and advantages of the present invention will be readily understood after reading the following detailed description together with the claims.

### DETAILED DESCRIPTION OF THE INVENTION

In the following, a detailed representative embodiment of the present invention will be described.

A lubricant of the present invention can be used in the processing of a metallic material. Further, examples of the processing may include press working, e.g., press forming, blanking, fine blanking, piercing, bending, burring, drawing, trimming and crimping. The lubricant may contain a paraffinic hydrocarbon (a saturated chain hydrocarbon) as a main ingredient (a lubricant base). The paraffinic hydrocarbon in this embodiment may includes liner hydrocarbons (normal paraffin), branched chain hydrocarbons (isoparaffin) and cyclic hydrocarbons (cycloparaffin). The paraffinic hydrocarbon may preferably have a carbon number of 8-13. That is, the paraffinic hydrocarbon may preferably have the formula, CₙH₂ₙ₊₂, wherein n is an integer from 8-13. In particular, the paraffinic hydrocarbon may be at least one of the compounds selected from the group of octane (C₈), nonane (C₉), decane (C₁₀), undecane (C₁₁), dodecane (C₁₂), tridecane (C₁₃) and isomers thereof. As will be recognized, the paraffinic hydrocarbon having a carbon number of 8-13 may be liquid at ambient temperature.

Generally, the paraffinic hydrocarbon may increase in boiling point with the carbon number. The paraffinic hydrocarbon having a carbon number of 14 or more may have an excessively high boiling point (i.e., an excessively slow evaporation rate). Therefore, such a higher paraffinic hydrocarbon is not preferable for a lubricant base or lubricant ingredient. In addition, the paraffinic hydrocarbon having a carbon number of 16 or more may generally be solid at ambient temperature. Therefore, the higher paraffinic hydrocarbon having a carbon number of 16 or more is not preferable for the lubricant ingredient. Conversely, the paraffinic hydrocarbon having a carbon number of 5-7 may have a sufficiently low boiling point (i.e., an allowable evaporation rate). Therefore, such a lower paraffinic hydrocarbon can be used as the lubricant ingredient. However, the paraffinic hydrocarbon having a carbon number of 5-7 is not generally preferable for the lubricant ingredient because it may generally produce a bad smell. Further, the paraffinic hydrocarbon having a carbon number of 1-4 may generally be gas at ambient temperature. Therefore, the paraffinic hydrocarbon having a carbon number 1-4 cannot substantially be used as the lubricant ingredient.

The paraffinic hydrocarbon may preferably have a boiling point of 210° C. or less, more preferably 160-200° C. The lubricant containing such a low boiling point paraffinic hydrocarbon can naturally and rapidly evaporate from the metallic material within several hours to 24 hours at ambient temperature and pressure. Therefore, the lubricant can be evaporated within 24 hours, which time period corresponds to a maximum permitted time period in which the processed metallic material must be transferred to a next step (e.g., a plating or coating step) in a manufacturing line. As a result, it is not necessary to stop the manufacturing line in order to dry the processed metallic material. Further, it is preferable that the lubricant can completely evaporate within 12 hours, more preferably within 6 hours, and most preferably within 2 hours, at ambient temperature and pressure. In other words, it is preferable that the lubricant has a reasonable evaporation speed.

The lubricant may preferably be formulated so as to have a kinetic viscosity of 2.0 or less at 40° C., more preferably 1.8 or less, and most preferably 1.5 or less. The present lubricant thus formulated can be effectively prevented from excessively adhering to the metallic material because of its increased flowability. As a result, the present lubricant does not substantially produce undesirable lubricant residues on the metallic material after the metallic material is processed. Further, the lubricant having the kinetic viscosity greater than 2.0 at 40° C. may have reduced flowability. Therefore, the lubricant may tend to excessively adhere to the metallic material. As a result, the lubricant may produce a considerable amount of lubricant residues on the metallic material after the metallic material is processed. In addition, the lubricant cannot be effectively evaporated from the metallic material. This means that the lubricant may have reduced self-removability. Further, it is preferred that the lubricant may have a kinetic viscosity of at least 1.0 in order to ensure required lubricity of the lubricant.

The lubricant may preferably be formulated so as to have a flash point of 40-90° C., a freezing point of −40° C. or less, and an ignition point of 240° C. or more. The lubricant having a flash point not less than 40° C. can be handled in safety at ambient temperature. Generally, the lubricant having a flash point less than 40° C. is highly flashable at ambient temperature. Therefore, such a lubricant cannot be safely handled, in particular, in summertime or in tropical or subtropical regions. Further, the lubricant having a freezing point not greater than −40° C. can be easily handled even in wintertime, in particular, in a cold region. That is, the lubricant having a freezing point greater than −40° C. may possibly freeze when used in the cold region. In addition, the lubricant having an ignition point not less than 240° C. can be handled in safety, because such a high ignition point lubricant may have a low risk of ignition by heat or sparks that is produced during processing of the metallic material.

The lubricant contains the paraffinic hydrocarbon at a rate of 96-100 wt % of total weight of the lubricant. Therefore, the lubricant has a high volatility so as to naturally and rapidly evaporate from the metallic material at ambient temperature and pressure. As a result, the present lubricant does not substantially produce undesirable lubricant residues on the metallic material after the metallic material is processed. Therefore, it is not necessary to wash the processed metallic material in order to remove the lubricant residues therefrom before the processed metallic material is further processed (e.g., plated or coated). In other words, even if the processed metallic material is directly plated or coated, the plated or coated metallic material may have a good plated or coated surface free from any defects (e.g., uneven portions and blisters).

The lubricant may further include an additive as an additional ingredient, if necessary. The additive may preferably be added to the lubricant at a rate of up to 4 wt % of total weight of the lubricant. Examples of the additive may include a mineral oil, a synthetic oil, a sulfuric extreme pressure agent, an oil-based agent, a rust inhibitive agent, an antioxidizing agent, a corrosion prevention agent, a coloring agent, an antifoaming agent and a fragrant material.

Examples of the mineral oil for use in this embodiment may include, but are not limited to, many kinds of oils that can be produced in a general petroleum refinery process. Such a petroleum refinery process may include the steps of distilling a crude petroleum under normal and reduced pressures so as to obtain a distillate, and further treating the obtained distillate via at least one of solvent deasphalting, solvent extraction, hydrocracking, solvent dewaxing, catalytic dewaxing, hydrorefining, sulfuric acid scrubbing and white earth treatment.

Examples of the synthetic oil are poly-α-olefins, α-olefin copolymers, poly butenes, alkyl benzenes, polyoxyalkyleneglycols, polyoxyalkyleneglycol ethers, silicone oils and other such compounds.

The sulfuric extreme pressure agent may preferably include various types of sulfuric compounds that can provide extreme pressure property. In other words, the sulfuric extreme pressure agent is not limited to special sulfuric compounds. Examples of the sulfuric extreme pressure agent are sulfurized fats, sulfurized fatty acids, sulfuric esters, sulfurized olefins, polysulfides, thiocarbamates, sulfurized mineral oils and zinc dialkyldithiophosphate (which will be referred to ZnDTP hereinafter). Further, it is preferable that the lubricant is formulated such that sulfur content in the formulated lubricant is not greater than 10 ppm of total weight of the lubricant. It has been found that if the sulfur content is greater than 10 ppm of total weight of the lubricant, the lubricant may possibly produce rust and stain on the metallic material.

Examples of the oil-based agent may include beef fat, lard, soy been oil, canola oil, rice bran oil, coconut oil, palm oil, palm kernel oil and hydrogenated products thereof.

The rust inhibitive agent is not limited to special compounds. Examples of the rust inhibitive agent may include calcium-based rust inhibitive agent, barium-based rust inhibitive agent and wax-based rust inhibitive agent. Examples of the antioxidizing agent may include amine series compounds and phenolic compounds. Examples of the corrosion prevention agent may include benzotriazols, tolyltriazols and mercaptobenzothiazoles. Further, the coloring agent may be various types of dyes and pigments.

The lubricant of the present embodiment may have beneficial effects in processing of the metallic material. For example, the lubricant may effectively increase processing accuracy of the metallic material when it is fed between the metallic material and a processing tool (a mold). Further, the lubricant may effectively reduce the friction between the metallic material and the processing tool. Generally, the lubricant may be applied to the metallic material by means of, for example, but are not limited to, a roller and a sprayer. In addition, the lubricant that is applied between the metallic material and the processing tool (the mold) may effectively protect the processing tool from rusting and damaging, thereby providing a prolonged working life of the processing tool.

Next, the metallic material used in the present embodiment will be described. Examples of the metallic material are rolled steel plates (e.g., stainless steel plates, alloy steel plates and carbon steel plates) that have been broadly used in many fields for manufacturing cars, architectural materials, white goods, electronic devices or other such articles. Preferably, the metallic material may be a rust-resistant rolled steel plate. Examples of the rust-resistant steel plate may include an electrogalvanized steel plate, a hot dip galvanized steel plate, a zinc-nickel alloy electroplated steel plate, an organic composite plated steel plate or other such steel plates. However, a more preferable rust-resistant steel plate may be the electrogalvanized steel plate. The electrogalvanized steel plate has been broadly used because of good surface smoothness, easy weldability, easy coatability, good workability and inexpensive availability. The electrogalvanized steel plate can be processed or formed to a desired shaped product (e.g., car parts) by the processing.

In this embodiment, the normal rolled steel plate is used as the metallic material, the acceptable thickness thereof may be not greater than 1.4 mm, more preferably not greater than 1.2 mm, and most preferably not greater than 1.0 mm. Conversely, the rust-resistant rolled steel plate is used as the metallic material, the acceptable thickness thereof may be not greater than 2.0 mm, more preferably not greater than 1.6 mm, and most preferably not greater than 1.4 mm. Generally, it is preferable that the metallic material may have a thickness of at least 0.1 mm. Further, it is preferable that the electrogalvanized steel plate may have a thickness of at least 0.4 mm as specified in Japanese Industrial Standard (JIS) G3313.

The examples of the lubricant of the present invention will now be described. Further, the following examples are illustrative and should not be construed as limitations of the invention.

In a first test, two example lubricants (Examples 1 and 2) and five control lubricants (Controls 1-5) were prepared by utilizing the paraffinic hydrocarbons (the lubricant base) having various carbon numbers. Compositions of the two types of example lubricants (Examples 1 and 2) and the five types of control lubricants (Controls 1-5) are shown in Table 1.

TABLE 1

| | | | Containing | Containing | |
|---|---|---|---|---|---|
| | | | Rate of | Rate of | |
| | | Carbon | Lubricant Base | Additives | |
| | | Number | (wt %) | (wt %) | Types |
| Examples | 1 | 8-13 | 100 | 0 | Mixed Oil |
| | 2 | 10-13 | 100 | 0 | Mixed Oil |
| Controls | 1 | 4-12 | 100 | 0 | Mixed Oil |
| | | | | | (Gasoline) |
| | 2 | 8-16 | 100 | 0 | Mixed Oil |
| | | | | | (Kerosene) |
| | 3 | 10-26 | 100 | 0 | Mixed Oil |
| | | | | | (Light Oil) |
| | 4 | 13-16 | 100 | 0 | Mixed Oil |
| | 5 | 14-30 | 100 | 0 | Mixed Oil |

With regard to the lubricants of Examples 1 and 2 and Controls 1-5, lubrication performance was evaluated. In order to evaluate the lubrication performance, metallic materials (work pieces) having the lubricants were respectively processed or press worked (sheared or punched), so as to produce formed products (test pieces).

Preparation of the formed products was carried out under following conditions.

Processing Machine
AIDA pressing machine having a punch and a die (Aida Engineering)
Processing speed: 60 spm
Material of the punch: SKD11
Material of the die: SKD11

Work Pieces
SECC (JIS G3313; Steel Plates for General Purposes)
Width: 150 mm
Thickness: 0.3 mm

Application of the Lubricants
The lubricants of Examples 1 and 2 and Controls 1-5 were uniformly fed to the surfaces of the work pieces by a resin roll coater.

Processing (1)
The work pieces having the lubricants were respectively subjected to punching by the punch, thereby producing the formed products (test pieces) that have 3000 punched holes of 2.5 mm, 6.0 mm, 22 mm and 100 mm in diameter.

Processing (2)
The work pieces having the lubricants were respectively subjected to drawing by the punch, thereby producing the formed products (test pieces) that have 3000 draw portions of 2.5 mm in diameter.

After the processing (punching and drawing) of each of the work pieces was completed, the punch was visually observed for the surface appearance thereof, so as to determine occurrence of defects, including wear and damage. The appearance of the punch was evaluated based on the following evaluation standards:
A: Superior (No defects)
B: Fine or Good (Substantially no defects)
C: Poor (Minor defects)
D: Inferior (Significant defects)

In addition, each of the formed products thus formed was visually observed for the processed surface appearance of the punched holes and the draw portions, so as to determine occurrence of defects, including damage and burr. The processed surface appearance of the punched holes and the draw portions was evaluated based on the following evaluation standards:
A: Superior (No defects)
B: Fine or Good (Substantially no defects)
C: Poor (Minor defects)
D: Inferior (Significant defects)

Results are shown in Table 2.

TABLE 2

| | | Examples | | Controls | | | | |
|---|---|---|---|---|---|---|---|---|
| | | 1 | 2 | 1 | 2 | 3 | 4 | 5 |
| Processing | Appearance of Punch | A | A | B | A | A | A | A |
| (1) | Appearance of | A | A | B | A | A | A | A |
| | Processed Surface | | | | | | | |
| Processing | Appearance of Punch | A | A | B | A | A | A | A |
| (2) | Appearance of | A | A | B | A | A | A | A |
| | Processed Surface | | | | | | | |

As shown in Table 2, except for Control 1, the punch may have superior surface appearance in both of processing (1) and (2). This means that the lubricants of Examples 1 and 2 and Controls 2-5 may effectively prevent the punch from wearing during processing (punching and drawing). Also, except for Control 1, the punched holes and the draw portions of the formed products may have superior surface appearance. This means that the lubricants of Examples 1 and 2 and Controls 2-5 may form the punched holes and the draw portions free from damage and burr. These results demonstrate that the lubricants containing only the paraffinic hydrocarbons having carbon numbers of 8 or more may have excellent lubricity during processing. In other words, the lubricants containing the paraffinic hydrocarbons having carbon numbers less than 8 may have reduced lubricity.

In a second test, two example lubricants (Examples 1 and 2) and five control lubricants (Controls 1-5) were prepared in the same manner as the first test. Compositions of Examples 1 and 2 and Controls 1-5 are shown in Table 1.

With regard to the lubricants of Examples 1 and 2 and Controls 1-5, volatility or drying property was evaluated. In order to evaluate the volatility, drying time was measured with regard to each of metallic materials (work pieces) having the lubricants.

The second test was carried out under following conditions.

Work Pieces
SPCC (JIS G3141)
Length: 80 mm
Width: 60 mm
Thickness: 1.0 mm

Application of the Lubricants
The lubricants of Examples 1 and 2 and Controls 1-5 were uniformly applied to the surfaces of the work pieces.

Amount of the Lubricants
0.5 g for each of the work pieces

With regard to each of the work pieces, the drying time was measured. Measurement was performed in a condition that the work pieces were horizontally positioned in air.

Results are shown in Table 3.

TABLE 3

| | Examples | | Controls | | | | |
|---|---|---|---|---|---|---|---|
| | 1 | 2 | 1 | 2 | 3 | 4 | 5 |
| Drying Time (Hour) | 2 | 2 | 1 | 30 | =48 | 30 | =48 |

As shown in Table 3, the lubricants of Controls 2-5 may have considerably long drying time (more than 24 hours). To the contrary, the lubricants of Examples 1 and 2 and Control 1 may have extremely short drying time (within 2 hours). This means that the lubricants of Examples 1 and 2 and Control 1 may have extremely high volatility. These results demonstrate that the lubricants containing only the paraffinic hydrocarbons having carbon numbers not greater than 13 can naturally evaporated from the work pieces within 24 hours.

In a third test, two example lubricants (Examples 1 and 2) and five control lubricants (Controls 1-5) were prepared in the same manner as the first test. Compositions of Examples 1 and 2 and Controls 1-5 are shown in Table 1.

With regard to the lubricants of Examples 1 and 2 and Controls 1-5, odors emitted therefrom were evaluated. The odors were sensuously evaluated with regard to the lubricants.

The third test was carried out under following conditions.

Test Samples of the Lubricants
The lubricants of Examples 1 and 2 and Controls 1-5 were respectively dispensed into glass beakers of 500 ml.

Amounts of the Dispensed Lubricants
100 ml for each of the beakers

Five or more persons independently smelled the test samples of the lubricants in the beakers, thereby evaluate the odors of the lubricants. The odor of each of the lubricants was evaluated based on the following evaluation standards:
A: Superior (Good smell)
B: Fine or Good (Sharp smell but not bad smell)
C: Poor (Sharp bad smell)

Results are shown in Table 4.

TABLE 4

| | Examples | | Controls | | | | |
|---|---|---|---|---|---|---|---|
| | 1 | 2 | 1 | 2 | 3 | 4 | 5 |
| Evaluation of Odors | A | A | C | B-C | B-C | A | A |

As shown in Table 4, the lubricants of Examples 1 and 2 may have good smell. However, the lubricants of Controls 1-3 do not have agreeable smell. These results demonstrate that the lubricants containing only the paraffinic hydrocarbons having carbon numbers of 8-13 may have good smell. In other words, some of the lubricants containing the paraffinic hydrocarbons having carbon numbers less than 8 or greater than 13 may have bad smell.

From the results of the first to third tests, the lubricants containing the paraffinic hydrocarbons having carbon numbers of 8-13 at a rate of 100 wt % of total weight thereof may have excellent lubricity, good workability, quick-drying property and good odor property.

In a fourth test, an example lubricant (Example 1) was prepared in the same manner as the first test. Composition of Example 1 is shown in Table 1.

With regard to the lubricant of Example 1, lubrication performance of the lubricant was evaluated with regard to the metallic materials (work pieces) having different thicknesses. In order to evaluate the lubrication performance, the various thicknesses of metallic materials having the lubricant were respectively processed (sheared or punched), so as to produce formed products (test pieces).

Preparation of the Formed Products was Carried Out Under Following Conditions.

Processing Machine
AIDA pressing machine having a punch and a die (Aida Engineering)
Processing speed: 60 spm
Material of the punch: SKD11
Material of the die: SKD11

Work Pieces (1)
SPCD (JIS G3141; Steel Plates for Drawing)
Width: 100 mm
Thickness: 0.5 mm, 1.0 mm, 1.4 mm, 2.0 mm

Work Pieces (2)
SPCE (JIS G3141; Steel Plates for Deep Drawing)
Width: 100 mm
Thickness: 0.5 mm, 1.0 mm, 1.4 mm, 2.0 mm

Work Pieces (3)
SECD (JIS G3313; Galvanized Steel Plates for Drawing)
Width: 100 mm
Thickness: 0.5 mm, 1.0 mm, 1.4 mm, 2.0 mm

Work Pieces (4)
SECE (JIS G3313; Galvanized Steel Plates for Deep Drawing)
Width: 100 mm
Thickness: 0.5 mm, 1.0 mm, 1.4 mm, 2.0 mm

Application of the Lubricants
The lubricant of Example 1 was uniformly fed to the surfaces of the work pieces by a resin roll coater.

Processing
The work pieces having the lubricant were respectively subjected to drawing by the punch, thereby producing the formed products (test pieces) that have draw portions of 2.5 mm in diameter.

After the processing (drawing) of each of the work pieces was completed, the punch was visually observed for the surface appearance thereof, so as to determine occurrence of defects, including wear and damage. From the appearance, the punch was evaluated based on the same evaluation standards as the first test.

In addition, each of the formed products thus formed was visually observed for the processed surface appearance of the draw portions, so as to determine occurrence of defects, including damage and burr. From the observed appearance, the processed surface appearance of the draw portions was evaluated based on the same evaluation standards as the first test.

Results are shown in Table 5.

TABLE 5

| | | Thickness | | | |
|---|---|---|---|---|---|
| | | 0.5 mm | 1.0 mm | 1.4 mm | 2.0 mm |
| Work | Appearance of Punch | A | B | A | C |
| Pieces (1) | Appearance of | A | B | C | D |
| | Processed Surface | | | | |
| Work | Appearance of Punch | A | B | B | C |
| Pieces (2) | Appearance of | A | B | C | D |
| | Processed Surface | | | | |
| Work | Appearance of Punch | A | A | B | C |
| Pieces (3) | Appearance of | A | A | B | C |
| | Processed Surface | | | | |
| Work | Appearance of Punch | A | A | B | C |
| Pieces (4) | Appearance of | A | A | B | C |
| | Processed Surface | | | | |

As shown in Table 5, with regard to the work pieces (1) and (2) having thicknesses of 1.4 mm or less, the punch may have an acceptable surface appearance. Similarly, the draw portions of the formed products may have an acceptable surface appearance. This means that the normal steel plates having thicknesses of 1.4 mm or less can be reliably processed using the present lubricant. Also, with regard to the work pieces (3) and (4) having thicknesses of 2.0 mm or less, the punch may have an acceptable surface appearance. Similarly, the draw portions of the formed products may have an acceptable surface appearance. This means that the galvanized (rust-resistant) steel plates having thicknesses of 2.0 mm or less can be reliably processed using the present lubricant. It is considered that in the galvanized steel plates, the zinc film may function as a lubricant.

In a fifth test, an example lubricant (Example 1) was prepared in the same manner as the first test. Composition of Example 1 is shown in Table 1.

With regard to the lubricant of Example 1, influence of an additive contained in the lubricant on a post-processing (i.e., a coating) was evaluated. In order to evaluate the influence of the additive, various types of lubricants having different contents of the additive were prepared based on the lubricant of Example 1. The lubricants thus formulated were applied to the surfaces of the metallic materials (work pieces). Thereafter, work pieces having the lubricants were respectively processed (i.e., coated) after the lubricants were dried, so as to produce coated products (test pieces).

After the coating of the work pieces was completed, the coating surfaces of the test pieces were visually observed for the surface appearance thereof, so as to determine occurrence of defects including uneven portions and uncoated portions. From the appearance, the coated surfaces were evaluated based on the following evaluation standards:
A: Superior (No defects)
B: Fine or Good (Slight uneven portions)
C: Poor (Uncoated portions)

Results are shown in Table 6.

TABLE 6

| | Additive Content (wt %) | | | | | | | | | |
|---|---|---|---|---|---|---|---|---|---|---|
| | 0 | 1 | 2 | 3 | 4 | 5 | 6 | 7 | 8 | 9 |
| Surface Appearance | A | A | A | A-B | A-B | B | B | C | C | C |

As shown in Table 6, with regard to the lubricants containing the additive at a rate not greater than 4 wt %, the coating surfaces of the test pieces may have an acceptable appearance. However, with regard to the lubricants containing the additive at a rate of 5-9 wt %, the coating surfaces of the test pieces may have considerable uneven portions and uncoated portions. It is considered that such defects are caused by lubricant residues that are produced on the metallic material after the lubricants were evaporated. Further, with regard to the lubricants containing the additive at a rate not greater than 2 wt %, the coating surface of the test pieces may have excellent appearance. Therefore, it is considered that the lubricants containing the additive at a rate not greater than 2 wt % are more preferable.

A representative embodiment of the present invention has been described in detail. This detailed description is merely intended to teach a person of skill in the art further details for practicing preferred aspects of the present teachings and is not intended to limit the scope of the invention. Only the claims define the scope of the claimed invention. Therefore, combinations of features and steps disclosed in the foregoing detail description may not be necessary to practice the invention in the broadest sense, and are instead taught merely to particularly describe detailed representative examples of the invention. Moreover, the various features taught in this specification may be combined in ways that are not specifically enumerated in order to obtain additional useful embodiments of the present teachings.

What is claimed is:

1. A lubricant for use in press working of a metallic material, comprising:
a paraffinic hydrocarbon that is contained at a rate of 96-100 wt % of total weight of the lubricant,
wherein the lubricant has a kinetic viscosity of 2.0 or less at 40° C.

2. The lubricant as defined in claim 1, wherein the paraffinic hydrocarbon has a carbon number of 8-13 and a boiling point of 210° C. or less.

3. The lubricant as defined in claim 2, wherein when the metallic material is press worked while the lubricant is applied thereto, the lubricant remaining on the metallic material may naturally evaporate within 24 hours at ambient temperature.

4. The lubricant as defined in claim 1, wherein the metallic material comprises a rolled steel plate.

5. The lubricant as defined in claim 4, wherein the rolled steel plate comprises a rust-resistant rolled steel plate.

6. The lubricant as defined in claim 4, wherein the rolled steel plate has a thickness not greater than 1.4 mm.

7. The lubricant as defined in claim 4, wherein the rolled steel plate has a thickness not greater than 2.0 mm.

8. A method for press working a metallic material using a processing tool, comprising the steps of:
feeding a lubricant between the metallic material and the processing tool,
wherein the lubricant comprises a paraffinic hydrocarbon that is contained at a rate of 96-100 wt % of total weight of the lubricant, and
wherein the lubricant has a kinetic viscosity of 2.0 or less at 40° C.

9. The method as defined in claim 8, wherein the paraffinic hydrocarbon has a carbon number of 8-13 and a boiling point of 210° C. or less.

10. The lubricant as defined in claim 9 further comprising press working the metallic material having the lubricant, wherein after the press working, the lubricant remaining on the metallic material may naturally evaporate within 24 hours at ambient temperature.

11. The lubricant as defined in claim 8, wherein the metallic material comprises a rolled steel plate.

12. The lubricant as defined in claim 11, wherein the rolled steel plate comprises a rust-resistant rolled steel plate.
